(19) United States
(12) Patent Application Publication
(10) Pub. No.: US 2016/0200792 A1
(43) Pub. Date: Jul. 14, 2016

(54) Purification Process for PTH

(71) Applicant: CADILA HEALTHCARE LIMITED, Ahmedabad, Gujarat (IN)

(72) Inventors: Sanjeev Kumar Mendiratta, Ahmedabad, Gujarat (IN); Sanjay Bandyopadhyay, Ahmedabad, Gujarat (IN); Avanish Kumar Singh, Ahmedabad, Gujarat (IN)

(73) Assignee: Cadila Healthcare Limited, Ahmedabad, Gujarat (IN)

(21) Appl. No.: 14/912,984

(22) PCT Filed: Aug. 21, 2014
(86) PCT No.: PCT/IN2014/000539

§ 371 Date: Feb. 19, 2016

(30) Foreign Application Priority Data
Aug. 21, 2013 (IN) 2726/MUM/2013

(51) Int. Cl. C07K 14/635; B01D 15/36; C07K 1/18

(52) U.S. Cl. CPC C07K 14/635; C07K 1/18; B01D 15/363

(57) ABSTRACT

The present invention relates to improved method for purification of a recombinant parathyroid hormone (rhPTH¹⁻³⁴ or teriparatide), said process for purification of parathyroid hormone comprising following essential steps: (a) Enzymatic cleavage; (b) anion exchange chromatography, followed by other suitable purification steps; wherein step (a) and (b) can be carried out in any order.

### FIELD OF THE INVENTION

The present invention provides improved method for purification of a recombinant parathyroid hormone (rhPTH¹⁻³⁴ or teriparatide). The process of purification of PTH according to the present invention comprises use of an anion exchange chromatography in the first step prior to use of any cation exchange chromatography. Such process of purification results in highly purified rhPTH¹⁻³⁴, with more than 99% purity, without employing any HPLC column step in the process of purification.

### BACKGROUND OF THE INVENTION

Recombinant human parathyroid hormone (rhPTH¹⁻³⁴) or teriparatide is a biologically active N-terminal fragment of endogenous human parathyroid hormone (PTH). Therapeutically, teriparatide is used for the treatment of men and postmenopausal women with osteoporosis who are at high risk of fracture. It increases bone mineral density and reduces the risk of vertebral and non-vertebral fractures.

The inventors of the present invention have indigenously developed teriparatide by recombinant DNA technology using genetically engineered E. coli cells as host system. Teriparatide comprising 34 natural amino acids has a theoretical molecular weight of 4117.8 Da. Teriparatide is a cysteine-free polypeptide chain.

In human body, PTH is mainly synthesized and secreted by the chief cells of the parathyroid glands, as a 84 amino acids (9.5 kDa) containing single polypeptide chain. Upon release in to the blood stream, PTH binds to the specific membrane receptor mainly present in bone and kidney to maintain serum Ca²⁺ level. The hormone-receptor interaction leads to activation of both the cAMP-dependent protein kinase A and the calcium-dependent protein kinase C signaling pathways with a typical cascade system.

In circulation, the endogenous native PTH has a half-life of 2 to 5 min and more than 90% of its clearance is mediated by liver and kidney.

It has been observed through several biochemical and structural studies that the N-terminal 1-34 amino acids fragment of PTH produced recombinantly or synthetically remains fully active in receptor binding and its activation. For optimal receptor binding activity, the N-terminal portion, 1-27 amino acids of PTH¹⁻³⁴ polypeptide chain are found to be essential for biological activity. The N-terminal portion of PTH¹⁻³⁴ causes stimulation of cAMP upon binding to its receptor, whereas the C-terminal portion of PTH¹⁻³⁴ helps in providing most of the binding energy without leading to cAMP activation.

PTH plays an important role in Ca²⁺ homeostasis. Release of PTH is triggered from parathyroid cells via a plasma membrane bound calcium sensor, when concentration of Ca²⁺ is low in circulating blood (hypocalcaemia). If the hypocalcaemia is sustained, then hypertrophy and hyperplasia of the parathyroid gland occur. On the other hand, an increased concentration of Ca²⁺ in plasma inhibits the release of PTH by a negative feed-back mechanism.

The present invention is related to purification of recombinant PTH. There are several purification processes known in prior art. Such purification processes include use of high performance liquid chromatography (HPLC) which is expensive and requires a large amount of organic solvent during operation The high cost of the instrument, requirement of flame-proof manufacturing plant and requirement of large amount of costly good quality organic solvents used as mobile phase are the major limitations in the case of purification of PTH by HPLC at industry scale.

WO2009019715 discloses two steps orthogonal purification process for rhPTH (1-34) comprising of cation exchange chromatography optionally followed by preparative chromatography selected from HIC or RP-HPLC to yield a target protein of >98% purity.

WO2003102132 relates to a method for protein purification that involves the combination of non-affinity chromatography with HPTFF.

An Indian application 2991/MUM/2010 discloses purification process of PTH comprising cation exchange chromatography and gel filtration chromatography.

The process described in the present invention for purification of PTH does not include any column chromatography wherein organic solvents are used as mobile phase or any HPLC column chromatography during purification process of the said polypeptide molecule. Thus, the present invention discloses a simple, cost-effective, highly scalable, industrially viable and environmentally favorable process of purification to obtain highly purified rhPTH¹⁻³⁴. The process of purification disclosed in the present invention can be used for purifying PTH from a crude mixture containing rhPTH¹⁻³⁴ generated by any process.

### SUMMARY OF THE INVENTION

The present invention provides a method for purifying the parathyroid hormone (PTH), preferably recombinant PTH.

In one aspect, the present invention provides a non-HPLC process for purification of PTH, preferably recombinant PTH comprising use of multiple chromatography steps in aqueous phase.

In another aspect, the present invention provides a non-HPLC process for purification of PTH comprising an anion exchange chromatography, as the first column for removal of impurities followed by cation exchange chromatography for further purification to obtain the desired polypeptide molecule in highly purified form.

In one preferred aspect, the present invention provides a purification process of PTH from a fusion-partner-protein complex after carrying out a site-specific cleavage to isolate the desired polypeptide chain of PTH from the complex.

In another preferred embodiment, the present invention discloses the use of a fusion partner protein complex, wherein the fusion partner is linked with PTH molecule through a signature sequence specific for enzymatic cleavage, so that upon cleavage, PTH molecule gets isolated from its N-terminal position. The fusion partner protein can be selected from a group of protein molecules, which are known to have pI values (theoretical) of 7.2 or less than that and does not appear to contain any signature sequence in the polypeptide chain similar to that of the sequence required for the specific cleavage reaction.

In a preferred embodiment, the present invention provides a process for purification of PTH, preferably recombinant PTH, comprising the following steps:
1. Site-specific cleavage
2. Weak anion exchange chromatography
3. Weak cation exchange chromatography
4. Strong cation exchange chromatography
5. Ultrafiltration and diafiltration
6. Weak anion exchange chromatography.

In a further embodiment, any of the column steps from step three to six can be carried out in any order.

In another embodiment, the enzymatic cleavage reaction may be carried out subsequent to the first anion exchange chromatography step.

The abbreviations used in the present description are defined below:

DEAE Sepharose: Diethylaminoethyl sepharose

CM sepharose: Carboxymethyl sepharose

HPLC: High performance liquid chromatography

RP-HPLC: Reverse phase—High performance liquid chromatography

HIC: Hydrophobic interaction chromatography

HPTFF: High performance tangential flow filtration

r-Enk: Recombinant Enterokinase

MWCO: molecular weight cut-off

WFI: Water for Injection

### BRIEF DESCRIPTION OF DRAWINGS

FIG. 1 depicts the chromatography profile of the first weak anion exchange column step employed in the purification process of rhPTH¹⁻³⁴. rhPTH¹⁻³⁴ product does not bind to the anion exchange matrix and comes out in the column flow-through-and-wash fraction. Tightly bound contaminating proteins are stripped off the column with higher salt concentration (500 mM NaCl).

FIG. 2 depicts the chromatography profile of weak cation exchange column employed in the purification process of rhPTH¹⁻³⁴. Upon binding to the matrix, rhPTH¹⁻³⁴ is eluted out of the column, differentially, in desired fractions (as indicated) with 200 mM NaCl gradient. Prior to elution, the column is washed with 150 mM NaCl in buffer.

FIG. 3 depicts the chromatography profile of strong cation exchange column employed in the purification process of rhPTH¹⁻³⁴. Following loading of the protein solution, the column matrix is washed with the equilibration buffer, first, and a second wash is performed with a higher conductivity than the equilibration buffer. Elution is carried out with a buffer having pH and conductivity higher than the second wash buffer. During elution, the desired fraction of rhPTH¹⁻³⁴ is collected, as indicated in the figure, for further processing.

FIG. 4 depicts the chromatography profile of the second weak anion exchange column step employed in the purification process of rhPTH¹⁻³⁴. rhPTH¹⁻³⁴ product does not bind to the anion exchange matrix and comes out in the column flow-through-and-wash fraction. Tightly bound residual contaminating proteins are stripped off the column at higher salt concentration (500 mM NaCl), as indicated.

FIG. 5 depicts the polypeptide profile of rhPTH¹⁻³⁴ recovered from the second weak anion exchange column by non-reducing SDS-PAGE. Upon resolving on gel, protein bands were developed by Ag-staining Single band purity of rhPTH¹⁻³⁴ is evident from the SDS-PAGE analysis. Removal of residual amount of contaminating protein has been shown in lane 3.

FIG. 6 depicts the purity of the purified Drug Substance of rhPTH¹⁻³⁴ by RP-HPLC. More than 99% purity of the principal peak of rhPTH¹⁻³⁴ is observed with the purified Drug Substance material of rhPTH¹⁻³⁴.

### DETAILED DESCRIPTION OF THE INVENTION

The present invention provides a non-HPLC purification process of PTH, preferably recombinant PTH (rhPTH¹⁻³⁴).

In one of the embodiments, the present invention provides a purification process of PTH comprising the use of an anion exchange chromatography, first, followed by subsequent use of other columns for purification of PTH from crude mixture. Crude mixture may include contaminating proteins, endogenous proteins, product related substances and other impurities in addition to the desired protein.

In one of the embodiments, the present invention provides a non-HPLC process for purification of PTH comprising multiple ion exchange column chromatography steps.

In one preferred embodiment, the present invention provides a purification process of PTH from soluble fusion-partner-protein-PTH complex, wherein PTH is linked with the fusion partner via a specific cleavage site. However, the present invention envisages purification of PTH from cells genetically transformed with a vector containing the genes specific for the fusion-partner protein-cleavage site-PTH complex synthesized by any conventional fermentation processes known in the art.

In a preferred embodiment, the purification of PTH from fusion-partner-protein-PTH complex is carried out with the following steps:

1. Enzymatic reaction to cleave PTH from soluble fusion partner-PTH complex present in crude mixture

2. Anion exchange chromatography

3. Cation exchange chromatography

4. Cation exchange chromatography

5. Ultrafiltration and diafiltration

6. Anion exchange chromatography.

In an embodiment, the enzymatic cleavage may be carried out subsequent to the first anion exchange chromatography step.

In another embodiment, steps three to six can be carried out in any order.

In a preferred embodiment, purification of PTH from a crude mixture comprising fusion-partner-protein-PTH complex is carried out with the following steps:

1. Enzymatic cleavage

2. Weak Anion exchange chromatography

3. Weak Cation exchange chromatography

4. Strong Cation exchange chromatography

5. Ultrafiltration and diafiltration

6. Weak anion exchange chromatography.

Downstream process for the purification of the PTH (rhPTH¹⁻³⁴) product comprises the following steps—
Cell disruption
Isolation of inclusion body mass from cell lysate
Solubilization of inclusion bodies
Separation of rhPTH¹⁻³⁴ from the fusion-partner-protein-PTH complex by enzymatic cleavage
Reconditioning
Removal of the fusion-partner protein by weak anion exchange chromatography
Purification by weak cation exchange chromatography
Purification by strong cation exchange chromatography
Ultrafiltration/diafiltration (UF/DF)
Purification by weak anion exchange chromatography
Buffer exchange by ultrafiltration/diafiltration
0.22 μm terminal filtration
Storage of the Drug Substance at or below −20° C.

In a preferred embodiment the upstream process is carried out as follows:

After harvesting the fermentation batch, E. coli cells are collected by centrifugation and resuspended in lysis buffer. Cells are disrupted by using a high pressure cell homogenizer to isolate the insoluble inclusion body mass from the lysate in the form of pellet. Isolated inclusion body mass is solubilized and is submitted to enzymatic reaction. Enzymatic cleavage of the desired PTH polypeptide chain from the fusion-partner-protein-PTH complex takes place in 5-6 h time, under suitable conditions. At the end of reaction, the reaction mixture undergoes a reconditioning step followed by column purification.

Chromatography Methods Used in the Present Invention:

Anion Exchange Chromatography—In anion exchange chromatography, stationary phase carries positive charge to which negatively charged proteins bind, while passing through the column matrix. For carrying out anion exchange chromatography according to the present invention, other anion exchangers which also can be used are selected from DEAE sepharose, Mono Q, Q sepharose, Q sepharose XL, Capto Q and the like. Anion exchanger DEAE sepharose has been used in the present invention.

Cation Exchange Chromatography—In cation exchange chromatography, stationary phase carries negative charge to which positively charged polypeptide molecules bind, while passing through the column matrix. In cation exchange chromatography, cation exchanger can be selected from SP-5PW, SP sepharose, MonoS, Bio-rex70, CM sepharose and the like. In the present invention, CM sepharose has been used as weak cation exchanger and SP-5PW has been used as strong cation exchanger in the specified steps.

RP-HPLC—Analytical RP-HPLC is performed by using a reversed phase C18 column saturated with 0.1% TFA in mobile phase A. Separation of rhPTH¹⁻³⁴ Drug Substance is conducted out with acetonitrile in TFA (mobile phase B) at a flow rate of 1 mL/min, 40° C.

In the present invention, no HPLC column step has been used for the purification of PTH product.

The preferred manner of purification of rhPTH¹⁻³⁴ according to the present invention is illustrated below, which should not be interpreted as limiting the scope of the invention in any way.

Step 1: Cell Disruption

After harvesting the cell mass from a 13±2 L fermentation broth (working volume) by centrifugation, cell pellet was suspended in Tris buffer of pH 8.0. Cells were disrupted by using a high pressure cell homogenizer between 900-1100 pressure bar with a single passage, under cold conditions (2° C.-15° C.).

Step 2: Isolation of Inclusion Body Mass from Cell Lysate

Inclusion body mass was isolated from cell lysate by centrifugation at 10,500 g×1 h under cold condition. Pelleted inclusion body mass was resuspended and washed with Tris buffer of pH 8.0 by centrifugation in the presence of low concentration of urea, preferably with 0.5-1 M urea, under reducing condition.

Step 3: Solubilization of Inclusion Body Mass

After washing, inclusion body mass was solubilized by 8 M urea in Tris buffer of pH 8.0, under reducing conditions, for 1 h at ambient temperature. Solubilized inclusion body mass was centrifuged at 10,500 g×1 h at 2° C.-8° C. Clear supernatant fraction containing soluble fusion-partner-protein-rhPTH¹⁻³⁴ complex with other contaminants was subjected to enzymatic cleavage of PTH from the fusion-partner complex.

Step 4: Separation of rhPTH¹⁻³⁴ from the Fusion-Partner-Protein Complex by Enzymatic Cleavage

Supernatant fraction containing the fusion-partner-protein-rhPTH¹⁻³⁴ complex and other contaminants, at 1-2 mg/mL (total protein) was treated with r-Enterokinase for 5-6 h at ambient temperature, under reducing conditions, for enzymatic cleavage. Enterokinase cleaved the fusion-partner-rhPTH¹⁻³⁴ complex at a specific site to release rhPTH¹⁻³⁴ from the protein complex. Enterokinase cleaved at the C-terminus Lys residue of the signature sequence, (Asp)₄Lys, which was present in between the fusion-partner-protein and rhPTH¹⁻³⁴ molecule. Enzymatic reaction was terminated at the specified time by acidification with the addition of acetic acid. The mixture was passed through a depth filter to separate the soluble fraction from insoluble matter or precipitates generated during acidification. Subsequent to acidification, the mixture was passed through a depth filter to recover the soluble protein fraction, predominantly, containing rhPTH¹⁻³⁴ in permeate.

Step 5: Reconditioning of the Soluble PTH¹⁻³⁴ after Cleavage

After depth filtration, the soluble protein fraction comprising rhPTH¹⁻³⁴ and other minor contaminants underwent a reconditioning step in terms pH adjustment in order to match to the next column step equilibration condition. pH of the solution was adjusted to 8.2 with Tris or NaOH solution.

Step 6: Weak Anion Exchange Column Chromatography

After reconditioning, the protein solution was passed through a weak anion exchange column to recover majority of the rhPTH¹⁻³⁴ product from the mixture in column flow-through-and-wash fraction. Uncleaved fusion-partner protein and other protein contaminants remained bound to the anion exchange column matrix, which were stripped off the column at higher conductivity. At this step, rhPTH¹⁻³⁴ product recovered in the flow-through-and-wash fraction was observed to exhibit more than 90% purity, as assessed by analytical RP-HPLC.

Details of the Anion Exchange Column Conditions:

Column dimension—13 cm (h)×20 cm (i.d.)

Column bed volume—4 L

Equilibration buffer: Tris-Cl, pH 8.2

Flow rate—28 to 47 cm/h

Column wash—Tris-Cl, pH 8.2 containing 500 mM NaCl

Column cleaning—0.5 N NaOH

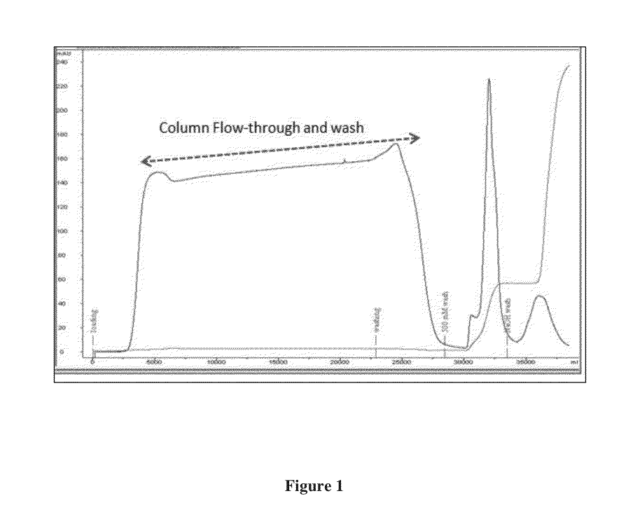

Chromatography profile of the weak anion exchange column step is illustrated in FIG. 1.

Step 7: Purification by Weak Cation Exchange Chromatography

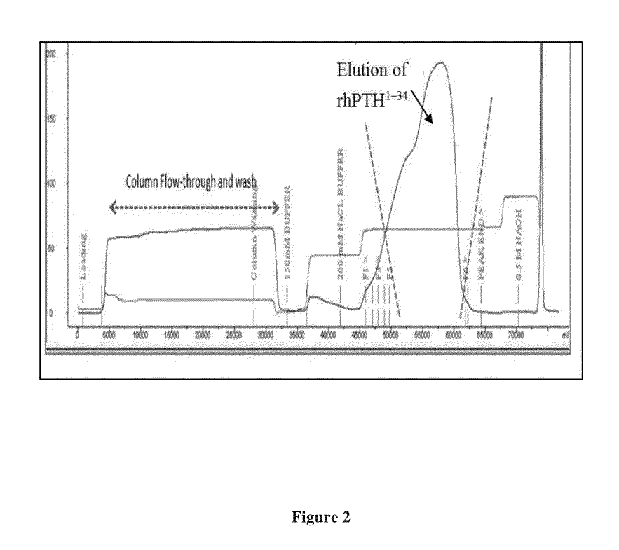

Following the weak anion exchange column chromatography step, rhPTH¹⁻³⁴ product was further purified by using a weak cation exchange column at pH 5.0 in bind-elute mode. This column step was performed mainly to remove the host cell derived contaminating products or non-product related impurities. Prior to loading on to the column, rhPTH¹⁻³⁴ solution was adjusted to pH 5.0 with the addition of diluted acetic acid. Upon binding to the column matrix, rhPTH¹⁻³⁴ product was eluted out of the column with 175-200 mM NaCl in a step-wise manner at the same pH. Prior to elution of rhPTH¹⁻³⁴, the column underwent an intermediate buffer wash with 150 mM NaCl. Chromatography profile of the weak cation exchange column step is illustrated in FIG. 2. After the weak cation exchange column step, eluted rhPTH¹⁻³⁴ shows more than 95% purity, as assessed by analytical RP-HPLC.

Details of the Weak Cation Exchange Column Conditions:

Column dimension—13 cm (h)×20 cm (i.d.)

Equilibration buffer: 20 mM Sodium acetate, pH 5.0

Column bed volume—4 L

Flow rate—47 cm/h

Elution—20 mM sodium acetate, pH 5.0 containing 175-200 mM NaCl

Column wash—Sodium acetate, pH 5.0 containing 250 mM NaCl

Column cleaning—0.5 N NaOH

Step 8: Purification by Strong Cation Exchange Chromatography

Weak cation exchange column-eluted fraction containing rhPTH¹⁻³⁴, further, underwent a third column step purification mainly for the removal of product-related substances by strong cation exchange column chromatography at pH 5.0. Column purification was performed at pH 5.0 in bind-elute mode. Subsequent to loading, the column was washed with 110 mM sodium acetate buffer of pH 6.2. Elution of rhPTH¹⁻³⁴ was carried out with 150 mM sodium acetate pH 7.2. rhPTH¹⁻³⁴ product eluted out of the column with a shoulder peak and was collected in fractions. Different peak fractions were analyzed by analytical RP-HPLC before pooling or selecting the desired fraction. Fractions containing more than 97% purity (by RP-HPLC) of the principal peak of rhPTH¹⁻³⁴ were pooled together for further processing.

Details of the Strong Cation Exchange Column Conditions:

Column dimension—23 to 26 cm (h)×10 cm (i.d.)

Column bed volume—2 L

Equilibration buffer: 20 mM Sodium acetate pH 5.0

Flow rate—92 cm/h

Elution—150 mM Sodium acetate, pH 7.2

Column cleaning—0.5 N NaOH

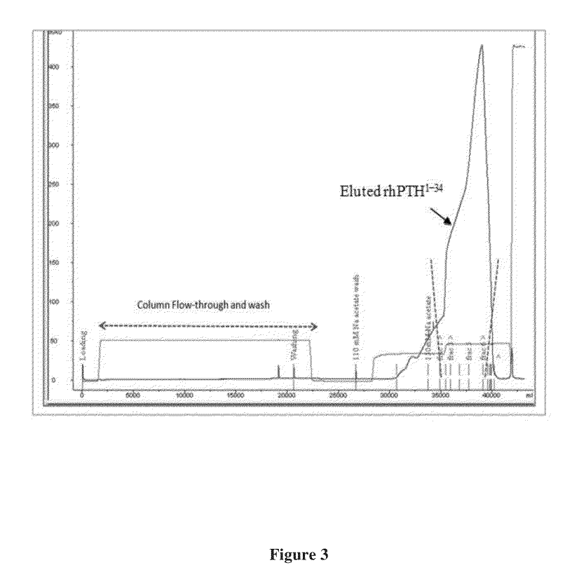

Chromatography profile of rhPTH¹⁻³⁴ elution from strong cation exchange column is illustrated in FIG. 3.

Step 9: Ultrafiltration-Diafiltration

Strong cation exchange column-eluted rhPTH¹⁻³⁴ solution underwent an ultrafiltration-diafiltration step in order to tune-up to the next column step equilibration buffer conditions by adjusting the conductivity and pH to about 1.5 (±1) mS·cm⁻¹ and 5.0, respectively. Constant volume diafiltration of rhPTH¹⁻³⁴ solution was carried out by using a 1 kDa or 2 kDa membrane with low ionic strength acetate buffer of pH 5.0, until conductivity and pH of retentate attains the same as of the initial diafiltration buffer. After diafiltration, pH of the rhPTH¹⁻³⁴ solution was adjusted to pH 8.2 with 1 M Tris-base (solution) in order to match to the next column step equilibration buffer pH.

Step 10: Purification by Weak Anion Exchange Chromatography

After diafiltration, rhPTH¹⁻³⁴ product solution was further passed through a weak anion exchange column for the removal of the residual amount of fusion-partner protein contaminants (product-related impurities). The desired rhPTH¹⁻³⁴ product was recovered in the column flow-through-and-wash fraction, whereas contaminating product-related substance(s) remain bound to the matrix.

Details of the Weak Anion Exchange Column Conditions:

Column dimension—25 cm (h)×10 cm (i.d.)

Column bed volume—2.5 L

Equilibration buffer—Tris-Cl, pH 8.2

Flow rate—28 cm/h

Column wash—Tris buffer with 500 mM NaCl, pH 8.2

Column cleaning—0.5 N NaOH

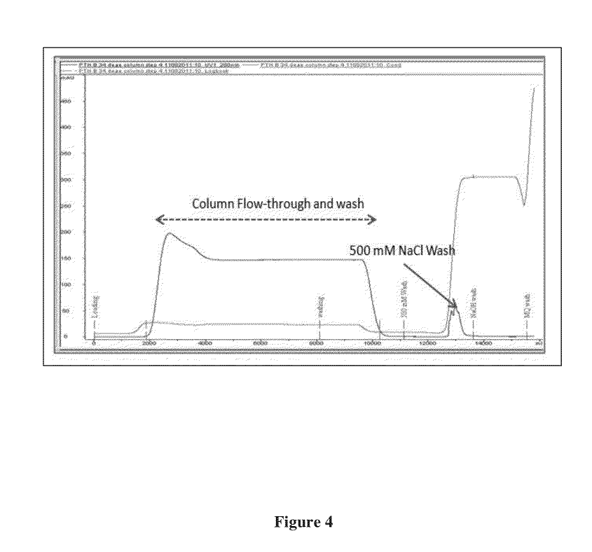

Chromatography profile of the weak anion exchange column step is illustrated in FIG. 4.

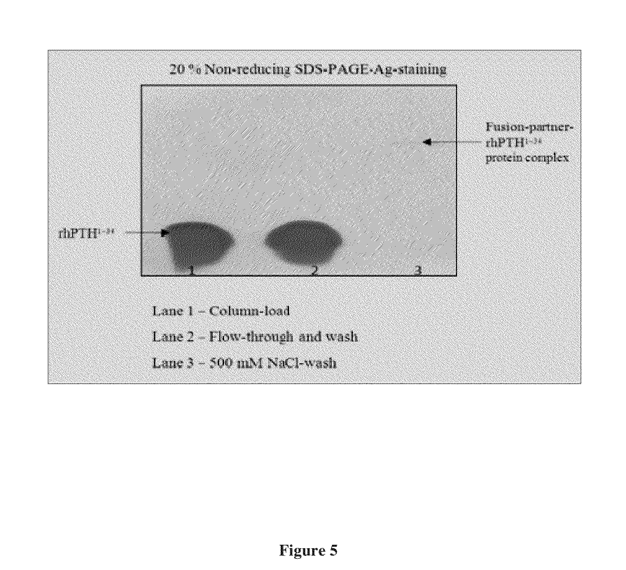

At this step, the purified rhPTH¹⁻³⁴ product recovered in the column-flow-through-wash fraction appears with a single broad band in gel, when analyzed by SDS-PAGE with Ag-staining, as shown in FIG. 5.

Step 11: Buffer Exchange by Ultrafiltration/Diafiltration

The desired rhPTH¹⁻³⁴ product solution recovered from the second anion exchange column step was mixed with acetic acid solution to adjust the pH to 5.0, first, and then submitted to ultrafiltration-diafiltration. Constant volume diafiltration is performed with sodium acetate buffer of pH 4.0 by using 1 kDa or 2 kDa MWCO membrane, under cold conditions (2° C.-15° C.), until pH and conductivity of retentate attain the same as that of the diafiltration buffer. This step was carried out to bring the purified rhPTH¹⁻³⁴ product in the drug substance storage buffer. Final concentration of the purified rhPTH¹⁻³⁴ product was maintained at around 1 mg/mL.

Step 12: 0.22 μm Terminal Filtration

After buffer exchange by ultrafiltration-diafiltration purified rhPTH¹⁻³⁴ product solution was passed through a 0.22 μm filter, aseptically and stored as frozen bulk Drug Substance of rhPTH¹⁻³⁴, at or below −20° C. in suitable storage container.

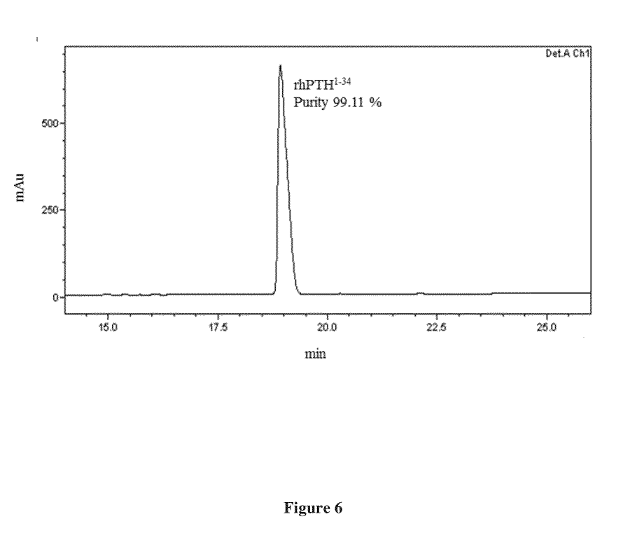

The final purified drug Substance of rhPTH¹⁻³⁴ exhibits more than 99% purity by analytical RP-HPLC shown in FIG. 6.

Results and Discussion

Thus the process of the present invention provides an efficient non-HPLC purification process of rhPTH¹⁻³⁴ from crude mixture. The said process results in highly purified preparation of rhPTH¹⁻³⁴ with more than 99% purity, as assessed by analytical RP-HPLC. Such highly purified preparation of rhPTH¹⁻³⁴ is considered to be suitable for therapeutic use in human after formulation as per conventional techniques known to a skilled person.

1. A process for purification of parathyroid hormone comprising:
(a) Enzymatic cleavage
(b) Anion exchange chromatography;
(c) Weak Cation exchange chromatography;
(d) Strong Cation exchange chromatography;
(e) Anion exchange chromatography
wherein
enzymatic cleavage can be carried out subsequent to the first anion exchange chromatography step,
step (c) to (e) can be carried out in any order.

2. The process as claimed in claim 1, wherein the enzymatic cleavage is carried out by recombinant enterokinase enzyme.

3. The process as claimed in claim 1, wherein the anion exchange chromatography is weak anion exchange chromatography.

4. The process as claimed in claim 1, wherein the anion exchanger is selected from DEAE sepharose, Mono Q and Q sepharose XL, preferably Q sepharose.

5. The process as claimed in claim 1, wherein the cation exchanger is selected from SP-5PW, SP sepharose, MonoS, Bio-rex70, CM sepharose.

6. The process as claimed in claim 1, wherein the cation exchanger for strong cation exchange chromatography is SP-5PW.

7. The process as claimed in claim 1, wherein the cation exchanger for weak cation exchange chromatography is CM sepharose.

8. The process as claimed in claim 1 further comprises ultrafiltration-diafiltration step subsequent to first anion exchange chromatography step.

9. The process as claimed in claim 8, wherein diafiltration medium is selected from phosphate buffer, acetate buffer, citrate buffer, succinate buffer and combination thereof.

10. The process for purification of parathyroid hormone as claimed in claim 1 from a crude mixture comprising the following steps:
(a) Cell disruption;
(b) Isolation of inclusion body mass from cell lysate;
(c) Solubilization of inclusion bodies;
(d) Separation of parathyroid hormone from the fusion-partner-protein-PTH complex by enzymatic cleavage;
(e) Reconditioning;
(f) Weak anion exchange chromatography;
(g) Weak cation exchange chromatography;
(h) Strong cation exchange chromatography;
(i) Ultrafiltration/diafiltration (UF/DF);
(j) Weak anion exchange chromatography;
(k) Buffer exchange by ultrafiltration/diafiltration;
(l) 0.22 m terminal filtration;
wherein,
enzymatic cleavage can be carried out subsequent to the first anion exchange chromatography step
step (g) to (l) can be carried out in any order

11. The process as claimed in claim 10, wherein parathyroid hormone is recombinant parathyroid hormone.

12. The process as claimed in claim 10, wherein parathyroid hormone is fused with fusion partner through cleavage site.
